(19) United States
(12) Patent Application Publication
(10) Pub. No.: US 2013/0330707 A1
(43) Pub. Date: Dec. 12, 2013

(54) METHOD AND APPARATUS FOR DOSING AND SUPPLY OF NUTRIENTS DERIVED FROM DEWATERING PROCESSES

(71) Applicant: AFS BIOOIL CO., (US)

(72) Inventors: Oleg Krifuks, South San Francisco, CA (US); Vadim Krifuks, South San Francisco, CA (US)

(73) Assignee: AFS BIOOIL CO., South San Francisco, CA (US)

(21) Appl. No.: 13/651,605

(22) Filed: Oct. 15, 2012

Related U.S. Application Data
(60) Provisional application No. 61/548,218, filed on Oct. 17, 2011.

(51) Int. Cl. C12Q 3/00

(52) U.S. Cl. CPC C12Q 3/00
USPC 435/3; 435/286.1

(57) ABSTRACT

A method and apparatus to combine the dewatering filtrate, biomass, and nutrients into a single stream for ease of homogenization, and then separate the products and feeds to be reused. The system comprises of an automated control system which monitors the nutrient levels in the effluent in real time, as well as the current levels within the biomass, an ultrasonic wave generator for homogenization, and a centrifuge for separation of the useful products. The method and automated control are a result of thorough analysis and research in the area of microorganism cultivation in a variety of phototrophic, heterotrophic, and hybrid bioreactors. By combining waste effluent with process streams in a bio facility, the electrical and chemical demands of a treatment step are negated.

### CROSS-REFERENCE TO RELATED PATENT APPLICATIONS

The present application claims priority to U.S. provisional application 61/548,218 filed on Oct. 17, 2011, the disclosures of which are incorporated by reference herein in their entireties.

### BACKGROUND OF THE INVENTION

The world is at a point of massive commercialization of biotechnologies that cultivate microorganisms for the production of many products, to include fuels, bioplastics, and chemicals. However, many microorganisms require specialized fertilizers to tailor the growth environment for the specific product, with common required elements being nitrogen, phosphorous, and potassium. As phosphorous is already showing signs of depletion in current harvesting methods, it is imperative that new methodologies are investigated to leave as much phosphorous as possible for other industrial uses, such as food production.

Current wastewater treatment practices divert their supernatant streams back to the headworks requiring additional energy and chemical costs to further process the stream. Wastewater streams are potentially very good sources for nutrient requirements for biotech processes.

### SUMMARY OF INVENTION

The embodiment relates to an improved method and apparatus for utilizing the nutrients in a wastewater stream for the growth in a bioreactor, such as that used for microorganisms, in a more energy efficient manner. The embodiment is employed to combine feed, waste, and product streams for the purpose of homogenization, and then separate product streams from those to be reused for use in any biotech process. The system uses ultrasonic homogenization and a combination of volume and weight dosing components to mix the incoming solution with micro and macro nutrients in order to balance the recipe based on the current properties of the supernatant, and the needs of the growing medium. This versatile system can be used by a wastewater treatment plant, or any biotech process, for cultivation of phototrophic, heterotrophic, and mesotrophic microorganisms.

The main benefit of our method and apparatus is that it reduces the power and chemical requirements for wastewater treatment processes, while remaining separate from the wastewater facility. This means that there is no expensive overhaul needed, nor potential risk to sensitive interior processes. Because the wastewater industry is conservative in adopting new methods, it is beneficial to not require interference with current best known practices.

The method of combination for homogenization is a result of the thorough analysis and research in the area of microorganism cultivation in a variety of phototrophic, heterotrophic, and hybrid bioreactors. By using the effluent in a beneficial manner instead of treating it, the electrical and chemical demands of a treatment step for the wastewater is negated.

The present embodiment has an advantage over competing systems in its ability to provide benefits for both wastewater treatment, and the biotech processes. When such a system is installed at a wastewater plant the supernatant from the dewatering step is no longer sent to the headworks for treatment. This elimination of a process means that the plant can reduce its electrical demand by up to 30%. This also means that the plant no longer has to purchase chemical additives in large quantities, leading to a reduced economic cost, and a reduced environmental impact.

### DESCRIPTION OF DRAWINGS

FIG. 1: A description of how the process will work is diagramed.

FIG. 2: A flow diagram of the method used for dosing and supply of nutrients derived from dewatering processes.

FIG. 3: A drawing of an apparatus for efficient dosing and supply of nutrients derived from the dewatering process of wastewater treatment plants for the cultivation of microorganisms.

The system analyzes the effluent for nutrients in real time, analyzing the amount of nutrients in the bioreactor, and adds nutrients as required for the cultivation of microorganisms. Dewatering supernatant is directed into our system, where it is analyzed for nutrient levels as decided by the operator. The system also analyzes the nutrient levels in the biotech process via time proportional composite samples, and implements the dosing. This implementation takes the form of a correcting solution stream, which is altered in real time as the monitoring of the bioprocess dictates.

This embodiment utilizes the standard real time probes outfitted with membrane sensors, optical sensors, ion selective electrodes, and standalone analyzers to monitor N, P, K, Ca, pH, dissolved gasses, conductivity, turbidity, etc. However, any number of elemental properties can be monitored via a like system.

Once a difference is calculated, the invention brings the nutrient levels up to the levels required by the operator. This is accomplished by adding these nutrients to the stream from stores. Ultrasound equipment is used to help dissolve and homogenize both the effluent nutrients, and the additional feeding elements into a concentrated fluid. By utilizing ultrasound equipment the speed of the dissolution process is increased by at least 100 times for highly soluble elements, and 10 to 30 times for difficult to dissolve elements. The limit for practically insoluble elements is increased by 5 to 30 times. The energy frequency of the ultrasound waves may be varied according to dissolution levels of all streams of the process and apparatus.

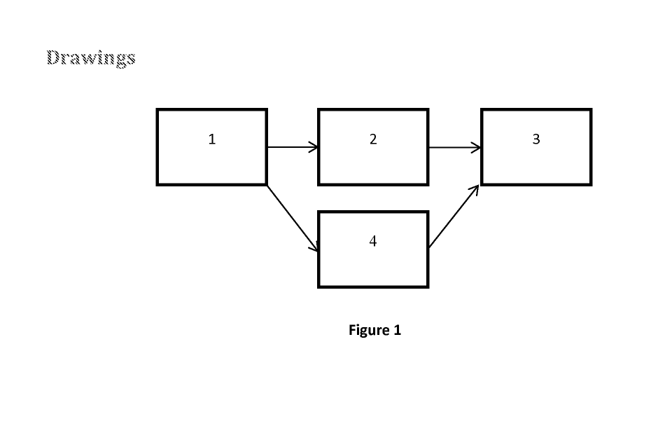

All of this is controlled via an automated system, which is accessible off site. A description of how the process will work is diagramed in FIG. 1.

1. Specific biotech cultivation requirements are determined by the plant operator. These cultivation requirements include any myriad number of properties, such as nutrient types, quantities, and dosing times.

2. The requirements determined in step 1 are entered into the automated system; and the system is set up to monitor these parameters within acceptable uncertainty ranges.

3. Selection of equipment and instrumentation for dosing and homogenization per step 1 requirements.

4. Assembly, startup, and testing of the automated dosing and homogenization system that beneficially uses a supernatant stream for supply of nutrients in a biotech process utilized for a microorganisms' cultivation.

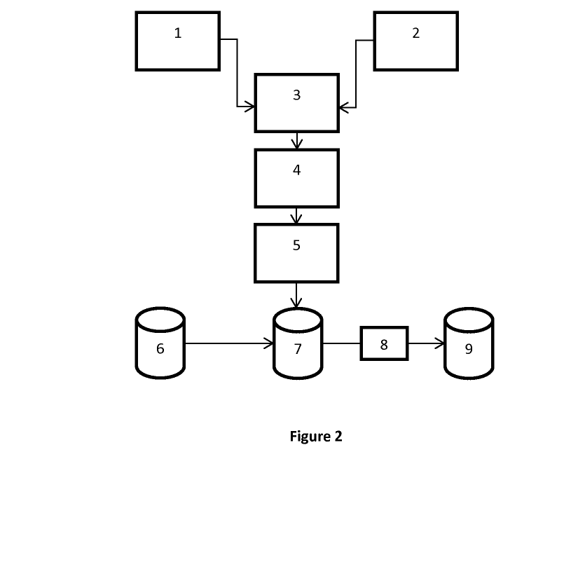

A detailed description of the process is diagramed in FIG. 2.

1. Automated real time analysis of supernatant nutrients. This is done online, but could also include grab sample analyzers and laboratory tests.

2. Automated real time analysis of conditions inside the biotech process. Additionally, analysis for the remaining feed elements and concentration of microorganisms.

3. Data logging and analysis from previous steps.

4. Computation of the required recipe needed to alter the supernatant to provide the optimum level of nutrients to the biotech process.

5. Volume and weight dosing of the supplementary stream utilizing ultrasound homogenization to ensure a uniform concentration of nutrients throughout the flow.

6. Intermediate storage tank for the accumulation and storage of supernatant.

7. Storage tank for feeding solution.

8. A control unit used to control specific nutrient feeding patterns. This could include parameters such as time, flow speed, feeding points, etc.

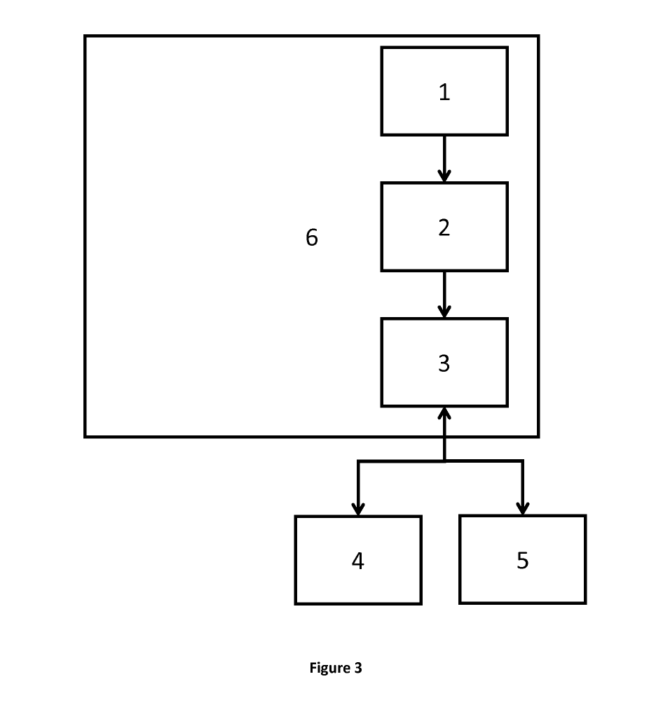

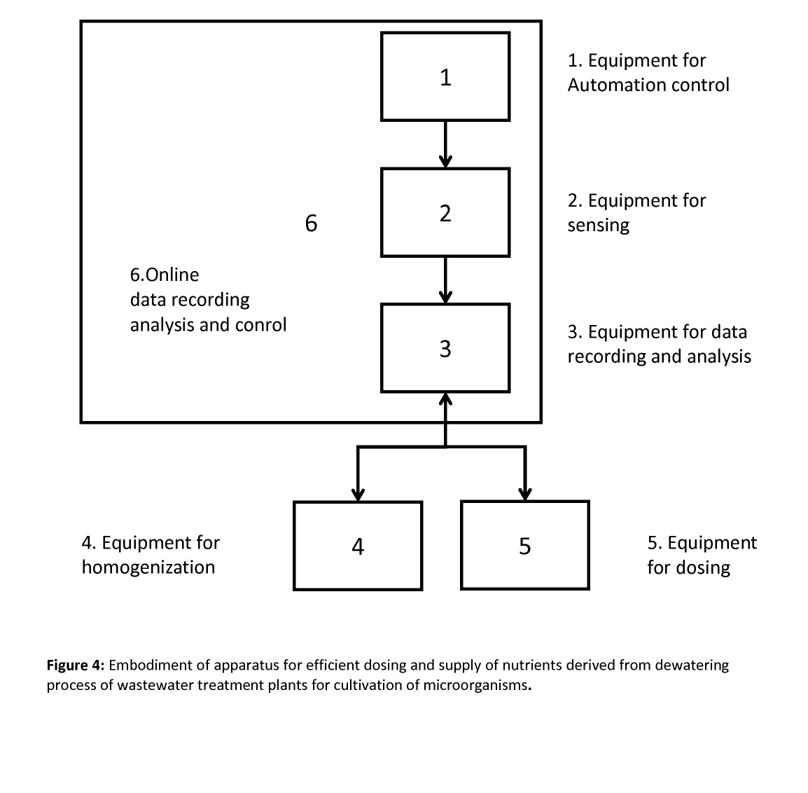

An embodiment detailed in FIG. 3, which contains the equipment block diagrams for the apparatus.

1. Equipment for automation control including input of parameters to a control system.

2. Equipment for sensing—a plurality of sensors.

3. Equipment for data recording and analysis—software, wireless, or over the internet.

4. Equipment for homogenization—to include an ultrasound generator.

5. Equipment for dosing—plurality of pumps to control flow rates throughout the apparatus.

6. Online data recording analysis and control—ability to monitor, analyze, and control the apparatus online.

1. A method for combining two processes, that of breaking down biomass and that of preparing nutrients, into a single process which reduces the power requirements needed for the homogenization of each independent process.

2. A method for efficient dosing and supply of nutrients derived from the dewatering process of a wastewater treatment plant, comprising:
i. Automatically computing a required recipe needed to alter the supernatant to provide the optimal level of nutrients to the biotech process.
ii. Automatically logging all pertinent data related to said supernatant dewatering filtrate and said set of nutrient supply and said set of cultivation properties in bioreactor cultivation.
iii. Automatically combining the supernatant dewatering filtrate, supplemental nutrients, and biomass into a single stream.
iv. Automatically homogenizing said supplemental stream, supernatant stream, and biomass with ultrasound to ensure a uniform concentration of nutrients throughout the flow of said stream.

3. A method as in claim 2 wherein the single stream containing supernatant, supplemental nutrients and biomass is separated into oil, biomass for further processing, and liquid feed by process of a centrifuge.

4. An apparatus as in claim 2 wherein the supernatant stream, supplemental stream, and biomass are combined into a single stream and homogenized by use of an ultrasonic wave generator, comprising:
i. Equipment for sensing, which will generate data/measurements, in communication with said automated control system throughout the apparatus, for measuring properties of all process streams in the apparatus, all holding tanks, all bioreactor cultivation process streams, all homogenization streams, both organic and inorganic.
ii. Equipment for data recording and analysis, in communication with the sensors, which will record all sensor measurements/data, and make calculations to take proper actions for the apparatus to work most efficiently.
iii. Equipment for homogenization, which may be distributed throughout all process streams in the apparatus, which will create uniform concentrations of all nutrients, organic and inorganic.
iv. Equipment for dosing connected to said process streams and said nutrient supply holding tanks, which will be controlled by the automated control system, to ensure adequate flow rates throughout all process streams of the apparatus to work most efficiently.

5. An apparatus as in claim 1, wherein the breakdown of inorganic materials takes place by application of ultrasonic waves generated from an ultrasonic wave generator for the purpose of homogenization.

6. An apparatus as in claim 1, 2, or 3 further comprising an ability to monitor and control the apparatus and its function over the internet by logging into a website.
